(19) United States
(12) Patent Application Publication
(10) Pub. No.: US 2016/0317560 A1
(43) Pub. Date: Nov. 3, 2016

(54) PARTICLES CONTAINING PHOSPHOLIPIDS OR BIOACTIVE FATTY ACIDS AND USES THEREOF

(71) Applicant: THE UNIVERSITY OF NORTH CAROLINA AT CHAPEL HILL, Chapel Hill, NC (US)

(72) Inventors: Joseph DeSimone, Chapel Hill, NC (US); Reid A. Roberts, Chapel Hill, NC (US); James Byrne, Carrboro, NC (US); Timothy Eitas, Hillsborough, NC (US)

(73) Assignee: The University of North Carolina at Chapel Hill, Chapel Hill, NC (US)

(21) Appl. No.: 15/035,162

(22) PCT Filed: Nov. 6, 2014
(86) PCT No.: PCT/US14/64312

§ 371 Date: May 6, 2016

Related U.S. Application Data
(60) Provisional application No. 61/901,105, filed on Nov. 7, 2013.

(51) Int. Cl. A61K 31/685; A61K 9/14

(52) U.S. Cl. CPC A61K 31/685; A61K 9/14; A61K 9/146

(57) ABSTRACT

The subject matter disclosed herein is directed to particles containing phospholipids and/or fatty acids and the uses thereof for treating autoimmune diseases, inflammatory diseases and for modulating immune and inflammatory responses. Methods of preparing the particles are also described.

### CROSS-REFERENCE TO RELATED APPLICATIONS

This application is a 371 filing of International Application No. PCT/US2014/064312 filed Nov. 6, 2014, which claims priority to U.S. Provisional Application No. 61/901,105, filed Nov. 7, 2013, the entire contents of which are incorporated herein by reference.

### FIELD OF THE INVENTION

The subject matter disclosed herein is directed to particles containing phospholipids and/or fatty acids and the uses thereof for treating autoimmune diseases and for modulating immune and inflammatory responses.

### BACKGROUND

Autoimmune diseases result from the immune system's failure to maintain self-tolerance to antigen(s) in the affected organ. There are at least a hundred known systemic and organ-specific autoimmune diseases. Among the organ-specific autoimmune diseases are multiple sclerosis, lupus, myasthenia gravis, thyroiditis, insulin-dependent diabetes mellitus, rheumatoid arthritis, psoriasis, Crohn's Disease, ulcerative colitis, and others.

Autoimmune diseases affect over 50 million people in the U.S. alone. These diseases are one of the top ten causes of death of women under the age of 65. These diseases are the number one cause of morbidity in women in the U.S. Over 100 billion dollars are spent on the management of autoimmune diseases in the U.S.

In spite of major and significant advances in molecular and cellular immunology in the last two decades, the molecular basis for self-tolerance and the mechanisms regulating it are still a major challenge in immunology, and autoimmune diseases remain a major medical problem. The immune-specific approaches to therapy of the disease, expected to be the most effective, have not yet yielded an effective therapy for any of the autoimmune diseases.

The subject matter disclosed herein addresses the shortcomings in the art with regard to the lack of effective treatments for many autoimmune diseases.

### SUMMARY OF THE INVENTION

In embodiments, the subject matter described herein is directed to phospholipid containing micro and/or nano-particles and their uses in treating autoimmune diseases.

In embodiments, the subject matter described herein is directed to fatty acid containing micro and/or nano-particles and their uses in treating autoimmune diseases.

In embodiments, the subject matter described herein is directed to a method of treating an autoimmune disease comprising administering phospholipid containing micro and/or nano-particles.

In embodiments, the subject matter described herein is directed to a method of treating an autoimmune disease comprising administering fatty acid containing micro and/or nano-particles.

In embodiments, the subject matter described herein is directed to a method of modulating levels of inflammatory cytokines in a subject by administering phospholipid containing micro and/or nano-particles.

In embodiments, the subject matter described herein is directed to a method of modulating levels of inflammatory cytokines in a subject by administering fatty acid containing micro and/or nano-particles.

In embodiments, the subject matter described herein is directed to a method of preparing phospholipid containing micro and/or nano-particles.

### DESCRIPTION OF THE DRAWINGS

FIG. 1A-D depicts the clinical courses of multiple sclerosis.

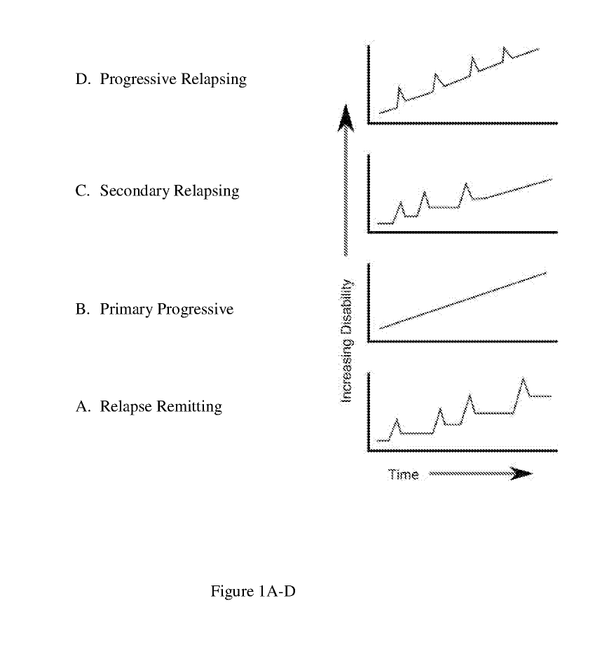

FIG. 2 depicts a mechanism for the development of transient immunosuppression and tolerance. Reproduced from Ho, P P. et al., Science TM; 4: 137ra73 (2012).

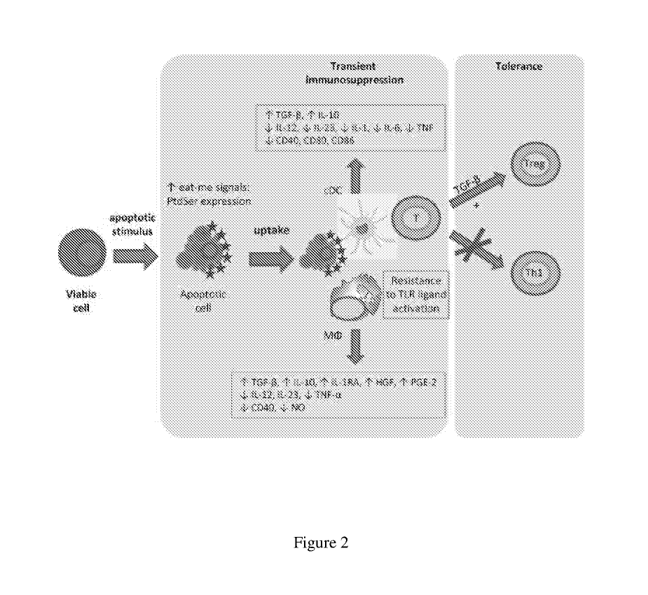

FIG. 3A-B are scanning electron micrographs of particles disclosed herein. FIG. 3A shows 80×320 nm blank PLGA particles. FIG. 3B shows 80×320 nm PS-loaded PLGA particles. PS=phosphatidylserine.

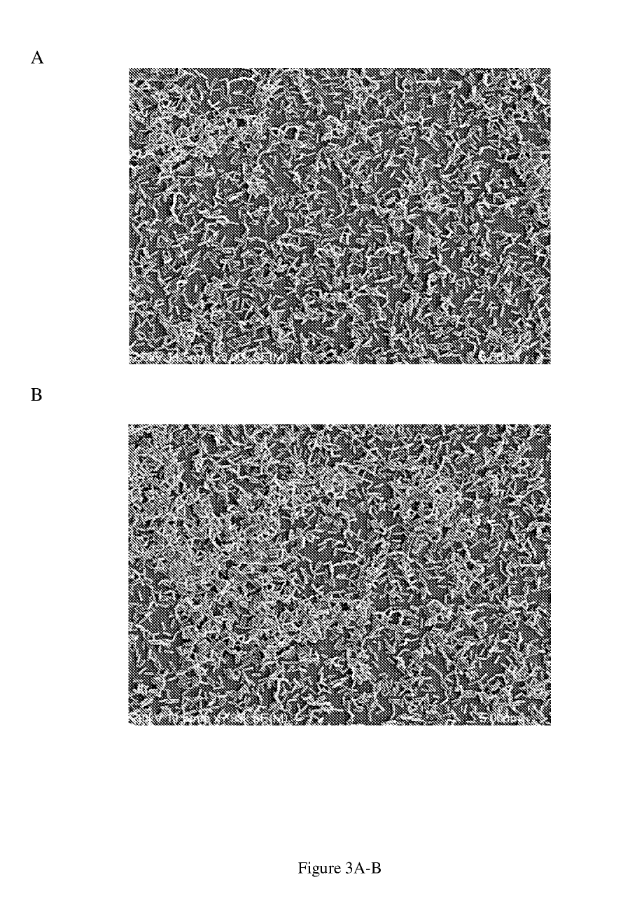

FIG. 4 is a graph depicting the release rate of phosphatidylserine from a representative particle disclosed herein.

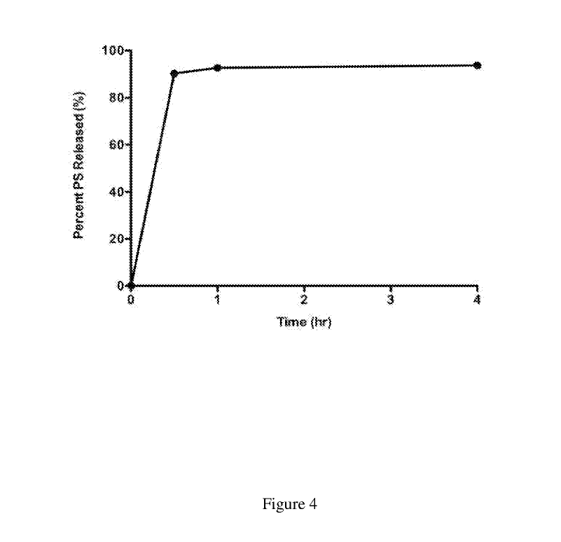

FIG. 5 depicts a murine model of multiple sclerosis (2D2) for evaluating T-cell activation in murine DC/T-cell co-culture.

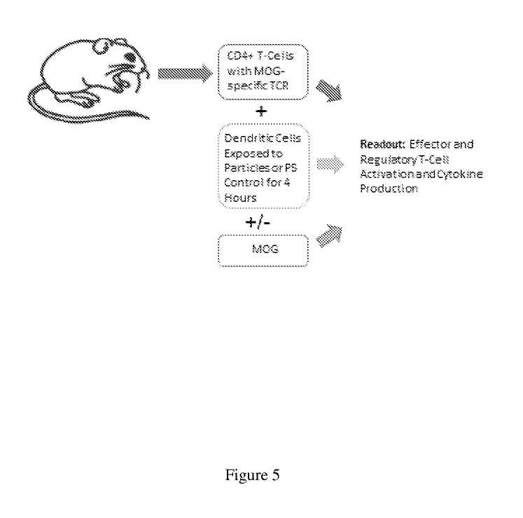

FIG. 6 presents data showing that PS-loaded PLGA particles reduce inflammatory cytokines. IFN-γ activates macrophages and Th₁ cells. As the data show, PS-loaded PLGA particles reduce this cytokine. As used throughout the figures, PS 12.5, PS 25 and PS 50 refer to the respective concentration of PS (μM) in a particle.

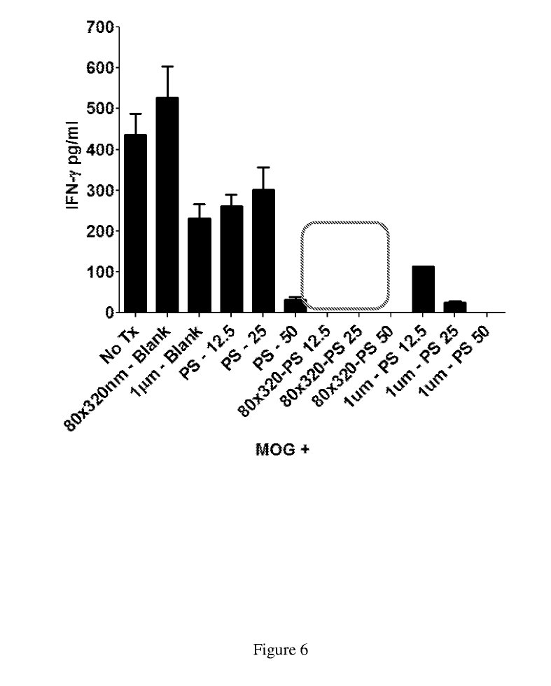

FIG. 7 presents data showing that PS-loaded PLGA particles reduce inflammatory cytokines. IL-2 stimulates growth of helper and cytotoxic T cells. As the data show, PS-loaded PLGA particles reduce this cytokine.

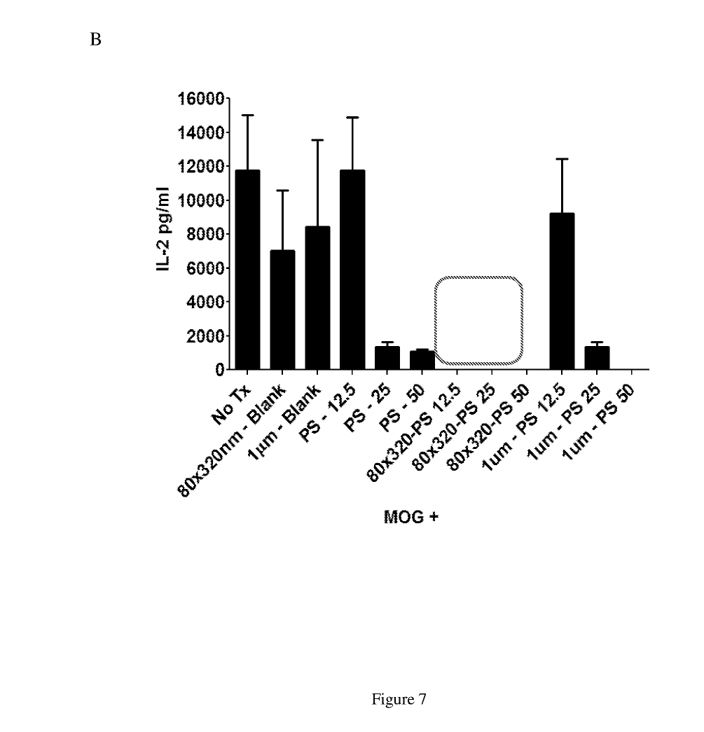

FIG. 8 presents data showing that PS-loaded PLGA particles reduce inflammatory cytokines. IL-6 is an endogenous pyrogen (produces fever). As the data show, PS-loaded PLGA particles reduce this cytokine.

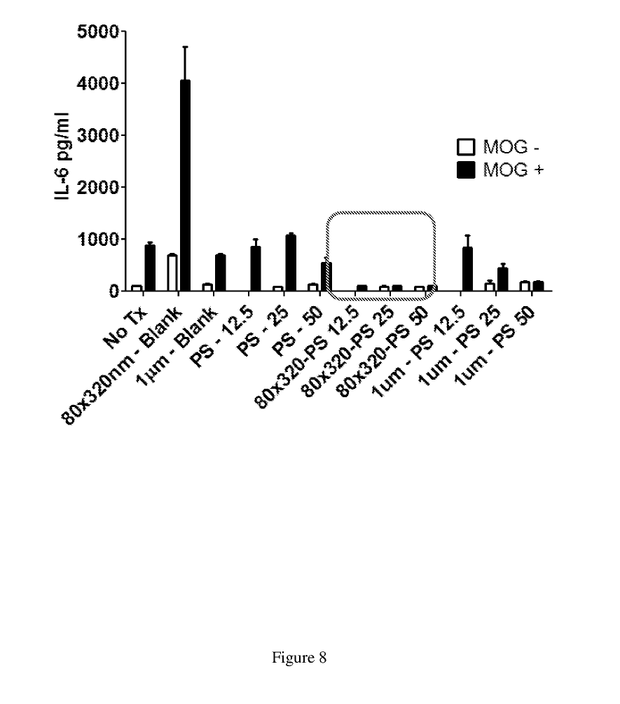

FIG. 9 presents data showing that PS-loaded PLGA particles reduce inflammatory cytokines. TNF-α mediates septic shock. As the data show, PS-loaded PLGA particles reduce this cytokine.

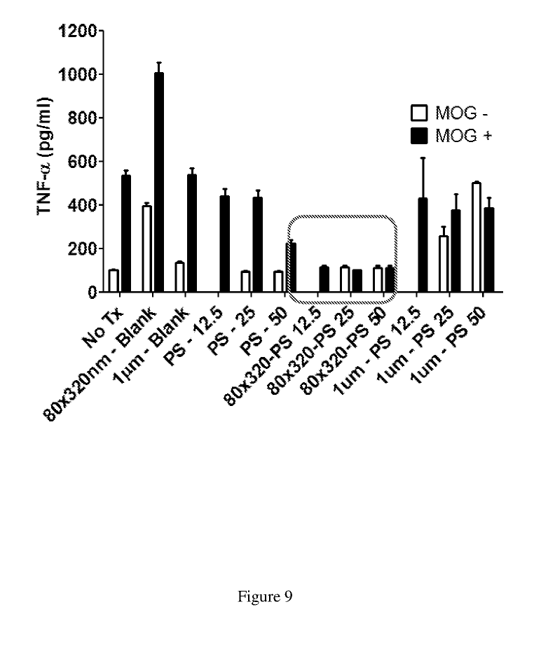

FIG. 10A-C presents data showing that PS-loaded PLGA particles induce T_{reg} population. BLK PAR=blank 80×320 nm particles; PS SOL=PS alone; PS PAR=PS-loaded 80×320 nm particles; FOXP3 is transcription factor in Tregs; IL-10 is an anti-inflammatory cytokine released by T_{regs}.

FIG. 11A-B depicts programming regulatory T cells with phosphatidylserine Nanoparticles. A) Flow plots of 2D2 T cells after co-stimulation with bone-marrow derived dendritic cells+/−the MOG peptide. Incubation with 50 μM of soluble phosphatidylserine (PS SOL) or 50 μM of phosphatidylserine encapsulated in 80×320 nm PLGA particles (PS PAR) is able to inhibit IFN-γ production by T cells in the presence of MOG, whereas blank 80×320 nm PLGA particles (BLK PAR) have no effect. PS PAR induce a significant increase in FOXP3+ T cells, not seen in the BLK PAR or PS SOL groups. B) Only particulate delivery of PS (PS PAR) are capable of inducing significant percentages of IL-10 and FOXP3 double positive T cells. (*=p<0.05).

FIG. 12 depicts a method for evaluating human T-cell proliferation in MS model.

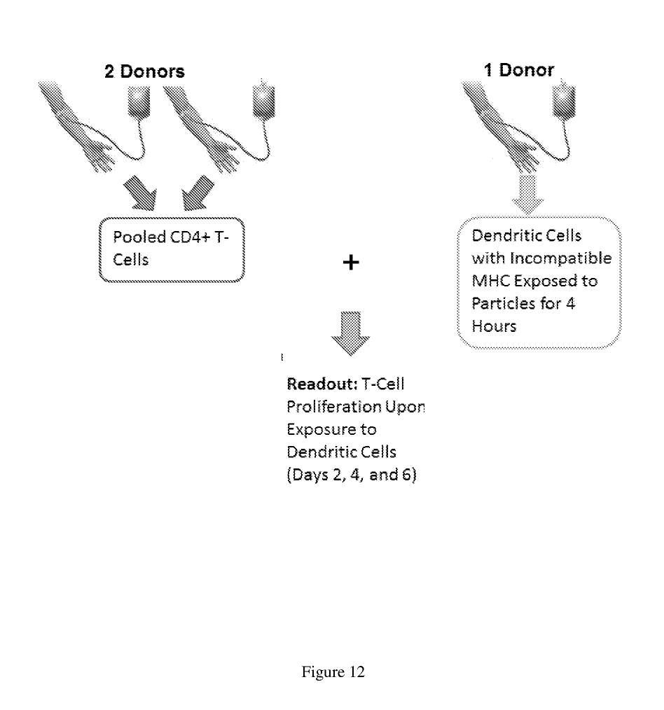

FIG. 13A-E provide data showing that PS-loaded particles reduce human effector T-cell proliferation.

FIG. 14 depicts differences between T-cells in the periphery and the CNS. Reproduced from Goverman J., Nature Reviews Immun.; 9: 393-407 (2009).

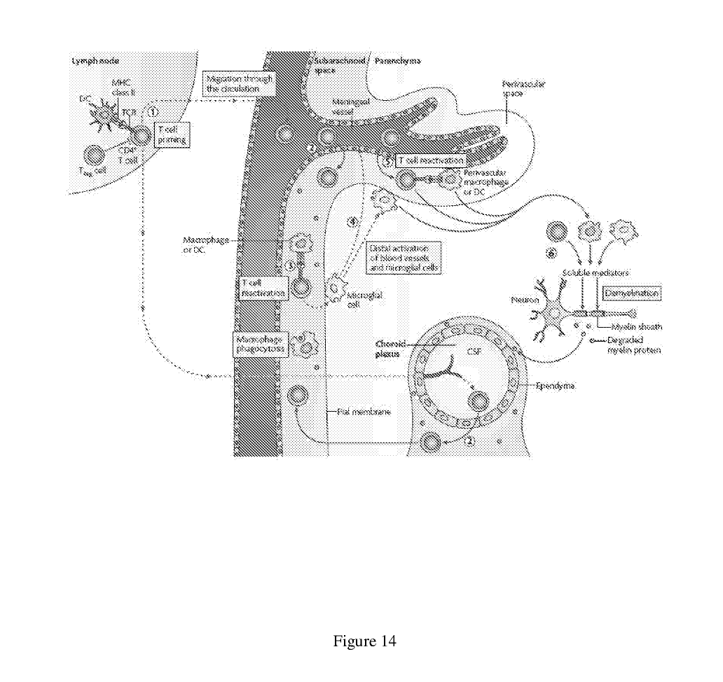

FIG. 15 depicts a murine model of primary progressive multiple sclerosis. Reproduced from the Reeves Foundation.

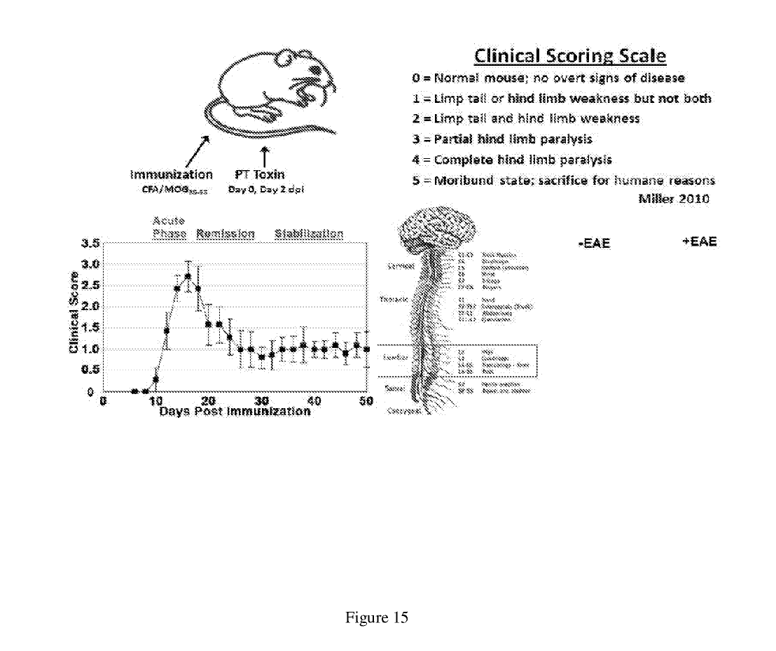

FIG. 16 provides data showing PS-loaded particles reduce MS symptoms. The study was 14 days to emphasize the suppression of the neuroinflammation ramp-up during the model. See Table 2.

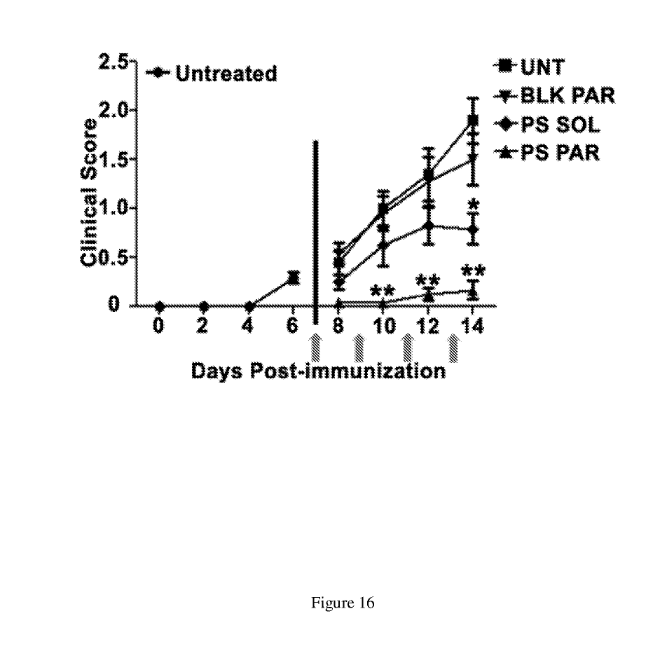

FIG. 17 depicts a murine model of relapse remitting multiple sclerosis.

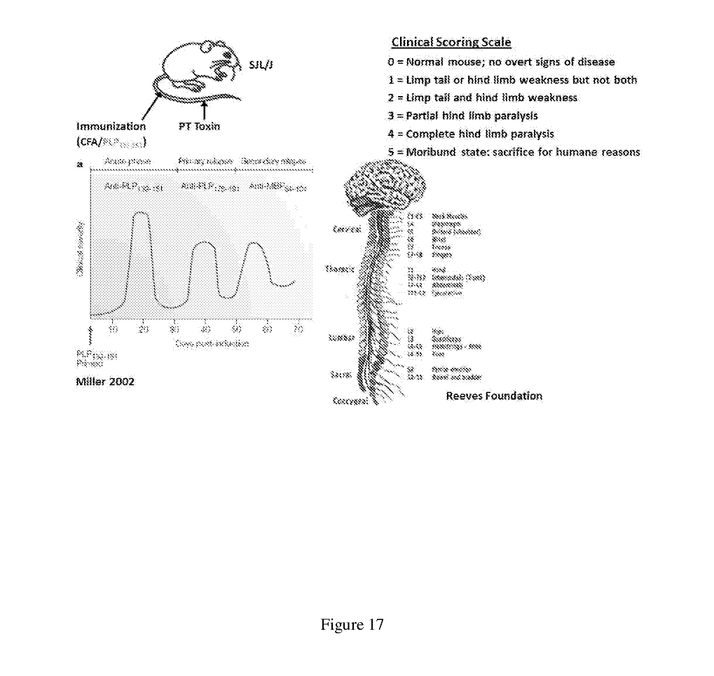

FIG. 18 depicts that in the relapse remitting model there is no statistical difference between treatments indicating that tail vein (peripheral) injection of particles is insufficient to treat a CNS disorder.

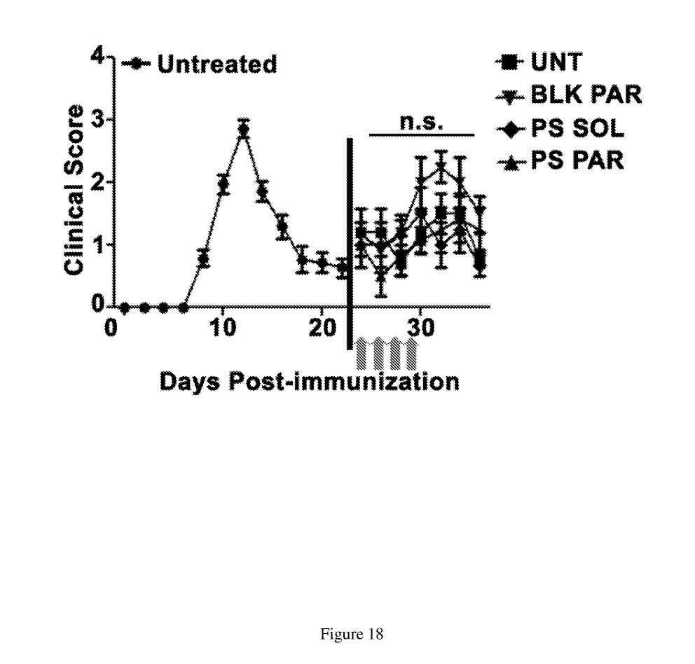

FIG. 19. Anti-inflammatory Programming Particles. A) Inflammasome activation in bone marrow derived mouse macrophages is significantly inhibited by PLGA nano- and microparticles containing the natural ligands phosphatidylserine (PS) and docosahexanoiec acid (DHA). Inhibition by DOTAP/DOPE coating is only effective at the 1 μm particle size.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Directed manipulation of biological signaling events, in particular those related to immune function, has the potential to transform how diseases are treated Immune networks are involved in the vast majority of all disease pathologies. Non-communicable diseases (NCD), e.g., cardiovascular disease, chronic respiratory disease, diabetes, and cancer, all have immune components that perpetuate disease. Current standards of care largely fail to effectively harness immune pathways. Clinical attempts to target the immune system are often crudely non-specific and unnatural, e.g., systemic immune suppression, systemic antibodies to immune receptors or cytokines and systemic cytokines. It is desirable to utilize the body's endogenous systems to restore itself to health.

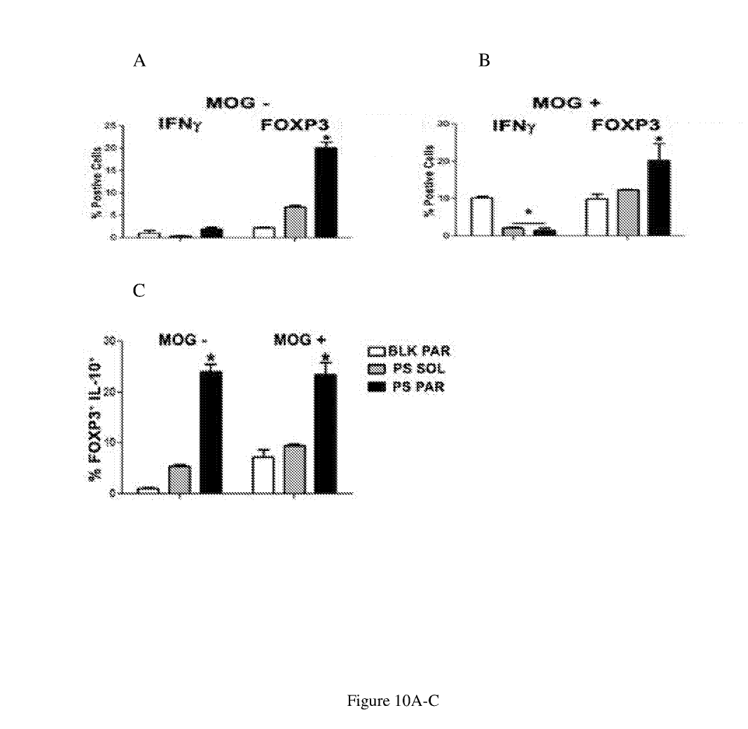

However, in many disease states and conditions, the immune system is providing a hyperreactive response that is disproportionate to the underlying cause as in the case of allergic reactions. Often times the immune response is undesirable as in the case of graft vs. host disease and in immune rejection of transplanted organs. Autoimmune inflammatory diseases are examples of undesirable immune responses whereby the effector-response of the immune system results in debilitating symptoms. It is therefore desirable to modulate such effector responses to produce a regulatory immune response to harness the body's endogenous immune system to restore health. While compounds are known that can dampen the immune response, described herein are particles that advantageously can provide unique properties to the particulated active agents and deliver the active agents to modulate immune responses. For example, as shown fully elsewhere herein, soluble phosphatidylserine (PS) can dampen IFN-γ production from T cells, while the same dose of PS delivered by a PLGA particle not only dampens IFN-γ production but also upregulates production of anti-inflammatory IL-10 and the FOXP3 transcription factor to generate significant populations of regulatory T cells (FIG. 10A-C). In other words, the particles containing the same dose amount of PS have emergent properties manifest when PS is delivered on a PLGA particle (80×320 nm and 1 μm have shown similar results). The active agents alone do not exhibit many of these properties.

Of particular interest are autoimmune diseases. Autoimmune disease states and conditions include a spectrum of autoimmune disorders ranges from organ specific diseases such as thyroiditis, insulitis, insulin-dependent diabetes mellitus, multiple sclerosis, iridocyclitis, uveitis, orchitis, hepatitis, Addison's disease, inflammatory bowel diseases (Crohn's disease, ulcerative colitis) and myasthenia gravis, to systemic illnesses such as rheumatoid arthritis, juvenile arthritis and systemic lupus erythematosus. Other disorders include immune hyperreactivity, such as allergic reactions and sepsis. Inflammatory diseases of chronic inflammation such as metabolic syndrome (obesity) are also treatable with the particles described herein. The particles are also useful in dampening the immune response when administered prior to or after organ transplantation. Of primary interest however is the class of autoimmune diseases involving T cells. However, it may also be possible to address autoimmune diseases primarily involving B cell exposure utilizing the particles described herein. The particles disclosed herein are particularly useful to treating these and other autoimmune disorders.

Described herein is subject matter directed to the fabrication of polymer micro- and nano-particles containing bioactive fatty acids and/or phospholipids. These classes of lipids are natural compounds generated by the body (or acquired through diet) that have potent biological signaling properties (Ho, P P, et al., Sci. Transl. Med., 4: 137 (2012); Kohli P., Br. J. Pharmacol., 158: 960-971 (2009); Nagy, L. et al., Physiol. Rev., 92: 739-789 (2012); Oh, D., Cell, 142: 687-698 (2010); Seki, H. et al., Scientific World Journal, 10: 818-831 (2010). Described herein, is the fabrication of these particles and testing showing their remarkable abilities to modulate immune responses.

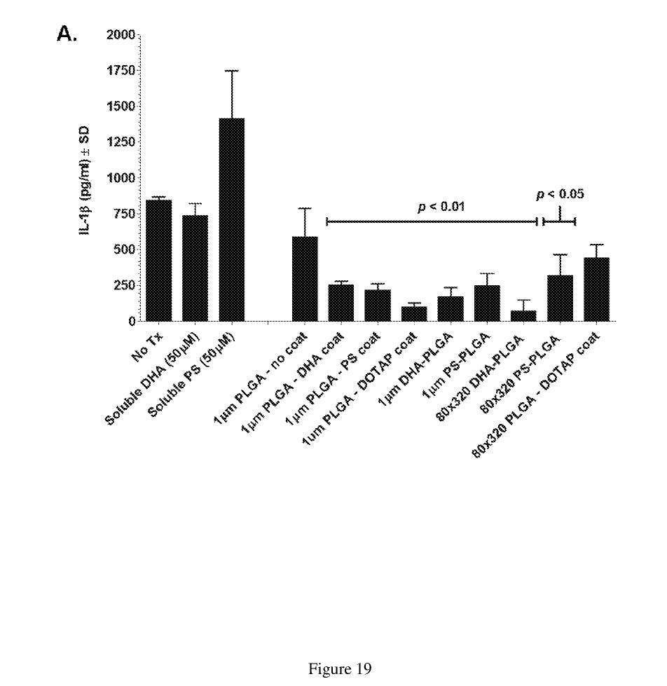

Disclosed herein are data showing emergent properties when bioactive fatty acids and phospholipids are delivered via the particles described herein. By way of example only, 1 μm and 80×320 nm DHA (docosahexanoic acid)-PLGA particles could significantly inhibit inflammasome signaling in murine macrophages with nano-sized particles trending to higher efficacy (FIG. 19). Delivery of the same dose of the bioactive fatty acid DHA in soluble form had no effect on inflammation. Particle fabrication did not destroy the bioactivity of DHA. Also as shown in FIG. 19, 1 μm and 80×320 nm PS-PLGA particles could inhibit inflammasome signaling in murine macrophages. Delivery of the same dose of PS in soluble form actually exacerbated inflammation, reflecting the surprising emergent properties that could not be predicted. Other phospholipids and bioactive fatty acids could have similar properties when particulated as described herein.

For the treatment of autoimmune diseases, it is desirable to deliver an effective amount of a phospholipid or a bioactive fatty acid. As used herein, the term “phospholipid” refers to compounds that include a non-polar lipid group and a highly polar end phosphate group. A particular family of phospholipid compounds is the phosphoglycerides. Another family is the sphingolipids. The term “phosphoglyceride” is used herein to describe compounds having a glycerol backbone, one or more lipid moieties and one or more of a phosphate end group, which are attached to the glycerol backbone. In sphingolipids, the backbone is a sphingosine. Most of the naturally-occurring phospholipids have two lipid moieties and one phosphate moiety attached to the glycerol backbone. As used herein, the term “lipid” describes a hydrocarbon residue having 2-30 carbon atoms. Lipids include natural or synthetic waxes, fatty alcohols, including their esters and ethers or any mixtures of same.

The term “bioactive fatty acid” or “fatty acid” as used herein refer to lipids shown to provide health benefits. These include the polyunsaturated fatty acids (PUFAs) and their derivatives, anmely the lipoxins and resolvins. In particular the cis-configuration of PUFAs. Butyric acid is an important fatty acid. Medium chain fatty acids contain 8-10 carbon atoms. These are mainly caprylic (C8:0) and capric (C10:0) acids. Long chain fatty acids contain 14 or more linearly arranged carbon atoms and may be saturated or unsaturated (with one or more double bonds). These fatty acids are found mostly as components of the triglycerides of edible oils and fats. Monounsaturated fatty acids include oleic acid, preferably in the cis-configuration. Omega-3 and omega-6 fatty acids include linoleic acid, linolenic acid, DHA, docosapentaenoic acid and eicosopentaenoic acid. The simplest omega-6 fatty acid is linoleic acid (C18:2), while linolenic acid (C18:3) is the simplest omega-3 fatty acid. Derivatives of biologic fatty acids such as lipoxin A4 are also included.

In naturally-occurring compounds, the lipids in phospholipids and glycerolipids are derived from fatty acids and are attached to the backbone via an O-acyl(ester) bond. The lipid moiety can be attached to the backbone either via an ether or an ester bond.

Phospholipids are common substances in biological systems. They make up the membrane in most cells in both plants and animals. These lipids are organized in double layer structures serving as barriers between the various compartments and providing the proper environment for receptors, enzymes and transport proteins. They also serve as transmitters for communication between cells. A large part of the human brain is made up of phospholipids. About 15% of the human brain phospholipids are phosphatidylserine (PS). Phosphatidylserine in a salt form has the following structure:

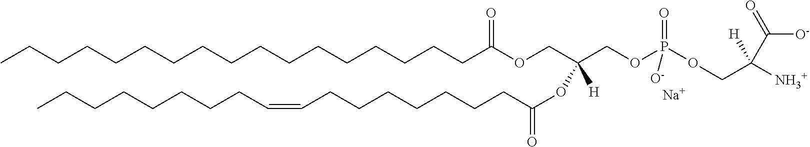 [SMILES: CCCCCCCC/C=C\CCCCCCCC(=O)O[C@H](COC(=O)CCCCCCCCCCCCCCCCC)COP(=O)([O-])OC[C@H]([NH3+])C(=O)[O-] ; [Na+]]

Other phospholids include phosphatidylcholine, phosphatidylinositols, phosphatidylethanolamines, phospatidylglycerol, and bisphosphatidyl glycerol. Useful fatty acids and phospholipids are disclosed in Kohli, P., Br. J. Pharmacol., 158: 960-971 (2009); Seki, H. et al., Scientific World Journal, 10: 818-831 (2010); Norling, L V. et al., J. Immunol., 186: 5543-5547 (2011); Serhan C N, Chem. Rev., 111: 5922-5943 (2011). A counterion is present when the phospholipid is in the salt form. Counterions include monovalent atoms.

In embodiments, phospholipid and/or bioactive fatty acid containing particles described herein provide efficient delivery of a phospholipid and/or bioactive fatty acid cargo to a biological target. Surprisingly, the phospholipids and/or bioactive fatty acid cargo associated with particles can provide enhanced response when compared with administration of the same amount of phospholipid or bioactive fatty acid in free form. Additionally, particle shape can provide enhanced responses at the same dose of phospholipid or bioactive fatty acid.

In a particular embodiment, a molded particle described herein comprises, i. a matrix of PLGA (85:15), having a molecular weight of about 75,000; and ii. phosphatidylserine, in an amount of from about 5 to about 75 μM. In preferred embodiments, these particles make up a plurality of monodisperse particles. A plurality refers to two or more essentially identical particles. In preferred embodiments, these particles are administered by pulmonary delivery as described elsewhere herein to treat autoimmune diseases.

Concentrations described herein such as molar (M) or micromolar (μM) can indicate the concentration of the material in the particle and/or the concentration of a solution used to make the particle.

As used herein, the term “particle” or “particles” is intended to mean one or more molded particles. The particles comprise a polymer matrix. It is within the matrix and/or on the surface of the matrix that the phospholipids are associated with the particle by electrostatic means. Methods of preparing particles are described in US 2011/0182805; US 2009/0028910; US 2009/0061152; WO 2007/024323; US 2009/0220789; US 2007/0264481; US 2010/0028994; US 2010/0196277; WO 2008/106503; US 2010/0151031; WO 2008/100304; WO 2009/041652; PCT/US2010/041797; US 2008/0181958; WO 2009/111588; and WO 2009/132206, each of which is hereby incorporated by reference in their entirety.

The phospholipid can be incorporated in a particle by forming a loading solution containing the phospholipid and a polymer or pre-polymer and preparing particles with the solution as described elsewhere herein. Once the solution is contacted with a mold, the solution is cured to form the particles. In this context, the term “cure” refers to chemical, heat or radiation crosslinking of a polymer. The polymer will crosslink to form a matrix.

A “polymer” that comprises the matrix of a particle refers to a chemical compound or mixture of compounds formed by polymerization and consisting essentially of repeating structural units. Useful polymers can be synthetic materials used in vivo or in vitro that are capable of forming the particles and are intended to interact with a biological system. Biodegradable, bioerodible and bioresorbable polymers are preferred. These terms refer to the polymers' susceptibility to decompose over time when in contact with a physiological medium such as a body fluid. In embodiments, the polymers will degrade at least 10% in about one minute to about 1 month. In embodiments, the polymers should be amenable to preparing a polymer solution that can be cured to form the polymer particles. Additionally, in embodiments, it is preferred that the identified polymer makes up essentially all of the matrix. In other words, the matrix consists of the identified polymer though there may be other trace components.

Polymers include, but are not limited to those taught in U.S. Pat. No. 5,514,378 (incorporated herein by reference). Biodegradable copolymers have also been described, including aliphatic polyester, polyorthoester, polyanhydride, poly alpha-amino acid, polyphosphagen, and polyalkylcyanoacrylate. Among aliphatic polyesters, polylactide (PLA), polyglycolide (PGA) and polylactideglycolide (PLGA). Biodegradable polymers include lactic acid polymers such as poly(L-lactic acid) (PLLA), poly(DL-lactic acid) (PLA), and poly(DL-lactic-co-glycolic acid) (PLGA). The co-monomer (lactide:glycolide) ratios of the poly(DL-lactic-co-glycolic acid) are preferably between 100:0 and 50:50. Most preferably, the co-monomer ratios are between 85:15 (PLGA 85:15) and 50:50 (PLGA 50:50). Blends of PLLA with PLGA, preferably PLGA 85:15 and PLGA 50:50, can be used. A particularly useful polymer is poly(lactic-co-glycolic acid) (PLGA).

The molecular weight of the PLGA can be any useful value. Of particular use are PLGA polymers having molecular weights from about 25,000 to about 100,000 daltons (g/mol). In embodiments, the PLGA polymers have a molecular weight of about 75,000 daltons.

Polymers include PEG. The term “PEG” or polyethylene glycol refers to an oligomer or polymer of ethylene oxide. PEG is often described by the molecular weight of the polymer chain. Useful chain lengths are described herein using common terminology. In embodiments, the PEG particles can be hydrogel particles.

The particle matrix comprises a polyethylene glycol (PEG) polymer. In embodiments, the polymers are water soluble. In embodiments, the matrix of the particle is a hydrogel. Hydrogels are formed by crosslinking polymer chains through physical, ionic or covalent interactions. A hydrogel is formed from a network of polymer chains wherein the network is water-insoluble.

PEG-based hydrogels are known. Useful PEG hydrogel particles are disclosed in U.S. Pat. No. 8,465,775, herein incorporated by reference in its entirety. Hydrogels suitable for use in the particles disclosed herein are preferably biocompatible, by which is meant that they are suitable to be introduced into a subject, i.e. they will not leach unwanted substances. Suitable hydrogels include macromolecular and polymeric materials into which water and small molecules can easily diffuse and include hydrogels prepared through the cross linking, where crosslinking may be either through covalent, ionic or hydrophobic bonds introduced through use of either chemical cross-linking agents or electromagnetic radiation, such as ultraviolet light, of both natural and synthetic hydrophilic polymers, including homo and co-polymers. Hydrogels of interest include those prepared through the cross-linking of: polyethers, e.g. polyakyleneoxides such as poly(ethylene glycol), poly(ethylene oxide), poly(ethylene oxide)-co-(poly(propyleneoxide) block copolymers; poly(vinyl alcohol); poly(vinyl pyrrolidone); polysaccharides, e.g. hyaluronic acid, dextran, chondroitin sulfate, heparin, heparin sulfate or alginate; proteins, e.g. gelatin, collagen, albumin, ovalbumin or polyamino acids; and the like. Because of their high degree of biocompatibility and resistance to protein adsorption, polyether derived hydrogels are preferred, with poly(ethylene glycol) derived hydrogels being particularly preferred. In embodiments, the hydrogels can have molecular weight cutoffs of, e.g., 200,000 daltons or more; 100,000 daltons; 50,000 daltons; 15,000 daltons; etc.

In some embodiments, the polymer is “PEG” or “poly(ethylene glycol)” as used herein, is meant to encompass any water-soluble poly(ethylene oxide). Typically, PEGs for use in the present invention will comprise the following structure: “—(CH₂CH₂O)ₙ—”. The variable (n) is 3 to 3000, and the terminal groups and architecture of the overall PEG may vary. PEGs having a variety of molecular weights, structures or geometries as is known in the art. “Water-soluble” in the context of a water soluble polymer is any segment or polymer that is soluble in water at room temperature. Typically, a water-soluble polymer or segment will transmit at least about 75%, more preferably at least about 95% of light, transmitted by the same solution after filtering. On a weight basis, a water-soluble polymer or segment thereof will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer or segment is about 95% (by weight) soluble in water or completely soluble in water.

An “end-capping” or “end-capped” group is an inert group present on a terminus of a polymer such as PEG. An end-capping group is one that does not readily undergo chemical transformation under typical synthetic reaction conditions. An end capping group is generally an alkoxy group, —OR, where R is an organic radical comprised of 1-20 carbons and is preferably lower alkyl (e.g., methyl, ethyl) or benzyl. “R” may be saturated or unsaturated, and includes aryl, heteroaryl, cyclo, heterocyclo, and substituted forms of any of the foregoing. When the polymer has an end-capping group comprising a detectable label, the amount or location of the polymer and/or the moiety (e.g., active agent) to which the polymer is coupled, can be determined by using a suitable detector. Such labels include, without limitation, fluorescers, chemiluminescers, moieties used in enzyme labeling, calorimetric (e.g., dyes), metal ions, radioactive moieties, and the like.

The polymer matrix can comprise crosslinkers. In some embodiments, the particles are composed of a crosslink density or matrix “mesh” density designed to allow slow release of the active agent. The term crosslink density means the mole fraction of prepolymer units that are crosslink points. Prepolymer units include monomers, macromonomers and the like. In some embodiments, the particles are configured to degrade in the presence of an intercellular stimulus. In some embodiments, the particles are configured to degrade in a reducing environment. In some embodiments, the particles contain crosslinking agents that are configured to degrade in the presence of an external stimulus. In some embodiments, the crosslinking agents are configured to degrade in the presence of a pH condition, a radiation condition, an ionic strength condition, an oxidation condition, a reduction condition, a temperature condition, an alternating magnetic field condition, an alternating electric field condition, combinations thereof, or the like. In some embodiments, the particles contain crosslinking agents that are configured to degrade in the presence of an external stimulus and/or a therapeutic agent. In some embodiments, the particles contain crosslinking agents that are configured to degrade in the presence of an external stimulus, a targeting ligand, and an active agent. In some embodiments, particles are configured to degrade in the cytoplasm of a cell. In some embodiments, particles are configured to degrade in the cytoplasm of a cell and release an active agent.

According to some embodiments, the particles can be controlled or time-release drug delivery vehicles. A co-constituent of the particle, such as a polymer for example, can be cross-linked to varying degrees and depending upon the amount of cross-linking of the polymer, another co-constituent of the particle, such as an active agent, can be configured to be released from the particle as desired. In certain embodiments, the particle includes a composition of material that imparts controlled, delayed, immediate, or sustained release of cargo of the particle or composition, such as for example, sustained drug release. According to some embodiments, materials and methods used to form controlled, delayed, immediate, or sustained release characteristics of the particles of the present invention include the materials, methods, and formulations disclosed in U.S. Patent Application nos. 2006/0099262; 2006/0104909; 200610110462; 200610127484; 2004/0175428; 2004/0166157; and U.S. Pat. No. 6,964,780, each of which is incorporated herein by reference in their entirety.

Any suitable amount of crosslinker can be employed, and the amount can be tailored depending on the desired properties of the matrix. When a crosslinker is present, the particles described herein comprise from about 2% wt. to about 40% wt crosslinker. In embodiments, the particles described herein comprise from about 4% wt. to about 30% wt crosslinker. In embodiments, the particles described herein comprise from about 5% wt. to about 25% wt crosslinker. In embodiments, the particles described herein comprise from about 6% wt. to about 20% wt crosslinker. In embodiments, the particles described herein comprise from about 7% wt. to about 15% wt crosslinker. In embodiments, the particles described herein comprise from about 8% wt. to about 12% wt crosslinker. In embodiments, the particles described herein comprise about 10% wt. crosslinker. In embodiments, a PEG monomer itself has a reactive end group on each end, such as an acrylate that acts as a crosslinker with other monomers to form the matrix. A non-limiting example is PEG-diacrylate.

The amounts of phospholipid and/or fatty acid that can be within the matrix of a particle are from about 0.01 wt. % to about 50 wt. %. In embodiments, the amount of phospholipid and/or fatty acid within the matrix of a particles is from about 0.1 wt. % to about 50 wt. %. In embodiments, the amount of phospholipid and/or fatty acid within the matrix of a particles is from about 1 wt. % to about 35 wt. %. In embodiments, the amount of phospholipid and/or fatty acid within the matrix of a particle is from about 1.5 wt. % to about 25 wt. %. In embodiments, the amount of phospholipid and/or fatty acid within the matrix of a particle is from about 1.5 wt. % to about 20 wt. %. In embodiments, the amount of phospholipid and/or fatty acid within the matrix of a particle is from about 12 wt. % to about 20 wt. %. In embodiments, the amount of phospholipid and/or fatty acid within the matrix of a particle is about 16 wt. %. In embodiments, the amount of phospholipid and/or fatty acid within the matrix of a particle is at least about 8 wt. %. In embodiments, the amount of phospholipid and/or fatty acid within the matrix of a particle is at least about 12 wt. %. In embodiments, the amount of a phospholipid and/or fatty acid within the matrix of a particle is at least about 16 wt. %. The term “drug loading” as used herein can refer to the concentration of the drug-containing solution that is used during the fabrication of the particle.

In embodiments, the loading solution can comprise about 1 to about 50% phospholipid (w/w). In embodiments, the loading solution can comprise about 2 to about 25% phospholipid. In another embodiment, the loading solution can comprise about 5 to about 15% phospholipid. In another embodiment, the loading solution can comprise about 10% phospholipid. In embodiments, the concentration of phospholipid in the loading solution correlates well to the loading of phospholipid in the resulting particle. Accordingly, the particles can comprise about 1 to about 50% phospholipid (w/w). In embodiments, the particles can comprise about 2 to about 25% phospholipid. In another embodiment, the particles can comprise about 5 to about 15% phospholipid. In another embodiment, the particles can comprise about 10% phospholipid.

The concentration of phospholipid and/or bioactive fatty acid in the particle is from about 1 to about 1,000 μM per particle. In embodiments, the concentration of phospholipid in the particle is from about 1 to about 100 μM per particle. In embodiments, the concentration of phospholipid in the particle is from about 5 to about 75 μM per particle. In embodiments, the concentration of phospholipid in the particle is from about 10 to about 60 μM per particle. In embodiments, the concentration of phospholipid in the particle is about 12.5, 25 or 50 μM per particle.

The particles are preferably molded wherein the molded particle further comprises a three-dimensional shape substantially mimicking the mold shape and a size less than about 50 micrometers in a broadest dimension. In further embodiments, the particles are preferably molded to have a three-dimensional shape substantially mimicking the mold shape and a size less than about 5 micrometers in a broadest dimension. Preferably, the molded particles have a first dimension of less than about 200 nanometers and a second dimension greater than about 200 nanometers. In other embodiments, particles less than 200 nanometers in each dimension provide improved immunomodulation effects. In some embodiments, the particles are less than 200 nanometers in each dimension. In other embodiments, the particles are less than 100 nanometers in a dimension.

Table 1 shows the particle characteristics of some embodiments of the particles described herein.

| Particle Type | Size (nm) | PDI | ZP (mV) | Loading |
|---|---|---|---|---|
| 80 × 320 nm | 208.3 | 0.104 | −6.4 | N/A |
| Blank PLGA | | | | |
| 80 × 320 nm PS- | 209.0 | 0.048 | −22.0 | 8.0% |
| loaded PLGA | | | | |

As referred to herein, an amount, value or shape that is the “same,” “substantially the same” or “substantially similar” is one that does not vary in a statistically significant way from a given reference point or value. With regard to particles formed by the present methods, the shapes and dimensions of the particles are reproducible and a plurality of particles is substantially identical. A plurality of particles means at least two particles. In embodiments, some insignificant artifacts may occur in some particles. In preferred embodiments, the particles are substantially identical. Scanning electron micrography can be used to evidence the substantially identical nature of the particles even at nanometer resolution. In all embodiments, the particles or a component(s) of the particle, such as an arm protruding from the body of the particle are configured and dimensioned to hinder phagocytosis of the particle by one or more macrophages.

As used herein, the term “substantially mimicking” means a molded particle that has a shape that is predetermined from the mold used to prepare the particle. This term includes variance in the shape, size, volume, etc. of the particle from the mold itself. However, the particles shape, size, volume etc. cannot be random since they are prepared from molds and substantially mimic the mold's shape, size, volume, etc. As used herein, the term “spherical” or “substantially spherical” refers to a shape that is a sphere or is a natural shape such as an emulsion particle that resembles a sphere or a dispersion process that yields a spherical particle. A “non-spherical” shape does not include the spherical or substantially spherical shapes. The term “amorphous” refers to a shape that is not engineered. A shape that is not prepared from a mold can be amorphous. Amorphous shapes by definition cannot be systematically reproducible. This is in contrast to molded shapes.

According to some embodiments, the composition can further include a plurality of particles, where the particles have a substantially uniform mass, are substantially monodisperse, are substantially monodisperse in size or shape, or are substantially monodisperse in surface area. In some embodiments, the plurality of particles have a normalized size distribution of between about 0.80 and about 1.20, between about 0.90 and about 1.10, between about 0.95 and about 1.05, between about 0.99 and about 1.01, between about 0.999 and about 1.001. According to some embodiments, the normalized size distribution is selected from the group of a linear size, a volume, a three dimensional shape, surface area, mass, and shape. In yet other embodiments, the plurality of particles includes particles that are monodisperse in surface area, volume, mass, three-dimensional shape, or a broadest linear dimension.

Particle characteristics used to describe the shapes examined include: a) the shape diameter (SD); it is the minimum diameter of a circumscribed circle around the particle; b) the minimum feature size (MFS); it is the diameter of the smallest distinct geometry of the shape; and c) the volume of the shape. All of these characteristics can be readily determined by one of skill in the art using the information disclosed herein and information known in the art. In embodiments where the shape of the particle is essentially a rod, the particles can have aspect ratios calculated by the width×height. Aspect ratios for rod shapes will be >1:1. In embodiments, the aspect ratio is 2:1; 3:1; 4:1; 5:1; 6:1; 7:1, 8:1; 9:1; 10:1 and so on.

In some embodiments, the physical properties of the particle are varied to enhance cellular uptake. In some embodiments, the size (e.g., mass, volume, length or other geometric dimension) of the particle is varied to enhance cellular uptake. In some embodiments, the charge of the particle is varied to enhance cellular uptake. In some embodiments, the charge of the particle ligand is varied to enhance cellular uptake. In some embodiments, the shape of the particle is varied to enhance cellular uptake. In some embodiments, the physical properties of the particle are varied to enhance biodistribution. In some embodiments, the size (e.g., mass, volume, length or other geometric dimension) of the particle is varied to enhance biodistribution. In some embodiments, the charge of the particle matrix is varied to enhance biodistribution. In some embodiments, the charge of the particle ligand is varied to enhance biodistribution. In some embodiments, the shape of the particle is varied to enhance biodistribution. In some embodiments, the aspect ratio of the particles is varied to enhance biodistribution. In some embodiments, the physical properties of the particle are varied to enhance cellular adhesion. In some embodiments, the size (e.g., mass, volume, length or other geometric dimension) of the particle is varied to enhance cellular adhesion. In some embodiments, the charge of the particle matrix is varied to enhance cellular adhesion. In some embodiments, the charge of the particle ligand is varied to enhance cellular adhesion. In some embodiments, the shape of the particle is varied to enhance cellular adhesion.

The particles disclosed herein are naturally perceived and processed by the immune system, in particular, the innate immune cells that are major effectors of immune function in all organs and disease states. These are monocytes, macrophages and dendritic cells (DC) in mouse and humans. While data suggest that blank/empty PRINT particles made of either PLGA or hydrogels in the nano to micron range do not engender immune responses in mice and humans, macrophages and dendritic cells have been shown herein to process a bioactive ligand differently depending on whether it is particulated in a particle as described herein. This might be due to formation of phagocytic synapses that enable receptor clustering when ligands for a receptor are presented in particulate fashion. Receptor clustering then triggers unique signaling pathways that are not activated by a soluble ligand. Therefore, when bioactive ligands are fabricated into particles, they exhibit emergent properties, at least in part through the ability to engage receptor clustering. By altering shape, size, composition and ligand density, we can modulate the kinetics, temporality, and intensity of biological responses.

In an embodiment, the subject matter disclosed herein is directed to a method of treating a subject comprising administering a particle as described herein. The particles can be administered in any appropriate pharmaceutical formulation.

Delivery of the particle to the target is described herein. As used herein, the term “deliver” refers to the transfer of a substance or molecule (e.g., phospholipid and/or bioactive fatty acid) to a physiological site, tissue, or cell. This encompasses delivery to the intracellular portion of a cell or to the extracellular space. As used herein, the term “intracellular” or “intracellularly” has its ordinary meaning as understood in the art. In general, the space inside of a cell, which is encircled by a membrane, is defined as “intracellular” space. Similarly, as used herein, the term “extracellular” or “extracellularly” has its ordinary meaning as understood in the art. In general, the space outside of the cell membrane is defined as “extracellular” space. It also includes bulk delivery of the particles by administering the particles to a particular target site or in a particular route of administration to a target site. Modes of administering the particles are described elsewhere herein. Mention is made of a particularly useful mode that is pulmonary delivery as described elsewhere herein.

Modulating particle size can correspond with differential uptake into lung cells. This particle design parameter may enable differential targeting to the intracellular and extracellular space throughout the body, a feature that could be exploited for specific therapeutic modalities. It is desirable to deliver cytokine- or immune-skewing compounds via particles locally to the lung or other regions (extracellular release, microparticle) while also delivering an antigen into innate immune cells (intracellular release, nanoparticle) so that the ensuing adaptive immune response could be skewed in a targeted manner. It has recently been shown that lymphoid T cells are licensed in the lung to enter either the central nervous system (CNS), intestines and pancreas, depending on the immune cues in the lung. The particles described herein can have sustained deposition in the lungs. Accordingly, the nanoparticles may be able to program T cells during this licensing phase with lung-resident particles to generate therapeutically skewed T cells in other organs (CNS, intestines, and pancreas).

In embodiments, the subject matter described herein is directed to a method of treating an autoimmune disease comprising administering an effective amount of the particles to a subject. As used herein, the term “subject” refers to a mammal, which means humans as well as all other warm-blooded mammalian animals. It is to be understood that the principles of the presently disclosed subject matter indicate its effectiveness with respect to all vertebrate species, including mammals, which are intended to be included in the terms “subject” and “patient.” In this context, a mammal is understood to include any mammalian species in which treatment is desirable, particularly agricultural and domestic mammalian species, such as horses, cows, pigs, dogs, and cats. As used herein “a subject in need thereof” may be a subject whom in an embodiment could have been diagnosed as suffering from the autoimmune disease intended to be treated.

Autoimmune diseases are described elsewhere herein. Mention is made of a particular autoimmune disease that is multiple sclerosis (MS), which is an inflammatory disease of the central nervous system (CNS) characterized by primary demyelination. It is believed to result from an autoimmune reactivity to myelin components. Multiple sclerosis is a chronic, neurological, autoimmune, demyelinating disease. Multiple sclerosis can cause blurred vision, unilateral vision loss (optic neuritis), loss of balance, poor coordination, slurred speech, tremors, numbness, extreme fatigue, changes in intellectual function (such as memory and concentration), muscular weakness, paresthesias, and blindness. Many subjects develop chronic progressive disabilities, but long periods of clinical stability may interrupt periods of deterioration. Neurological deficits may be permanent or evanescent.

The pathology of MS is characterized by an abnormal immune response directed against the central nervous system. In particular, T lymphocytes are activated against the myelin sheath of the neurons of the central nervous system causing demyelination. In the demyelination process, myelin is destroyed and replaced by scars of hardened “sclerotic” tissue which is known as plaque. These lesions appear in scattered locations throughout the brain, optic nerve, and spinal cord. Demyelination interferes with conduction of nerve impulses, which produces the symptoms of multiple sclerosis. Most subjects recover clinically from individual bouts of demyelination, producing the classic remitting and exacerbating course of the most common form of the disease known as relapsing-remitting multiple sclerosis.

Multiple sclerosis develops in genetically predisposed individuals and is most likely triggered by environmental agents such as viruses (Martin et al., Ann. Rev. Immunol. 10:153-87, 1992). It is believed that activated autoreactive CD4+T helper cells (Th1 cells) which preferentially secrete interferon-gamma (IFN-γ) and tumor necrosis factors alpha/beta (TNF-α/β), induce inflammation and demyelination in MS (Martin et al., Ann. Rev. Immunol. 10:153-87, 1992). It is believed that predisposition to mount a Th1-like response to a number of different antigens is an important aspect of MS disease pathogenesis. Proinflammatory cytokines (such as IFN-γ, TNF-α/β) and chemokines secreted by Th1 cells contribute to many aspects of lesion development including opening of the blood-brain-barrier, recruitment of other inflammatory cells, activation of resident glia (micro- and astroglia) and the effector phase of myelin damage via nitrogen and oxygen radicals secreted by activated macrophages (Wekerle et al., Trends Neuro Sci. 9:271-77, 1986).

Cytokines can be generally classified into 3 types: pro-inflammatory or inflammatory (IL-1α, β, IL-2, IL-3, IL-6, IL-7, IL-9, IL-12, IL-17, IL-18, TNF-α, LT, LIF, Oncostatin, and IFNc1α, β, γ); anti-inflammatory (IL-4, IL-10, IL-11, W-13 and TGF β); and chemokines (IL-8, Gro-α, MIP-1, MCP-1, ENA-78, and RANTES).

In embodiments, the subject matter disclosed herein is directed to methods of modulating the level of an inflammatory cytokine by administering to a subject an effective amount of particles disclosed herein. The term “modulating” refers to a change in the level of the cytokine when compared to a level prior to administering a particle as described herein. In embodiments, modulating a cytokine includes decreasing or reducing the expression or detectable level of a cytokine by at least 10%. This includes decreasing the level by about 20, 30, 40, 50, 60, 70, 80, 90, 95, 96, 97, 98, 99 or 100%.

In embodiments, the subject matter disclosed herein is directed to methods of ameliorating the symptoms of an autoimmune disease by administering to a subject an effective amount of particles disclosed herein. In embodiments, the subject matter described herein is directed to a method of ameliorating the symptoms of an autoimmune disease in a subject in need thereof. In embodiments, a subject in need thereof is a person diagnosed with an autoimmune disease. Symptoms of autoimmune diseases will, of course, depend on the particular disease. With regard to multiple sclerosis, prevalent symptoms include visual disturbances, tremors, dizziness, limb weakness, muscle spasms, numbness, loss of balance and coordination, mental changes, depression, paranoia and bladder and bowel dysfunction.

TABLE 2
PS-loaded particles reduce MS symptoms.

| | Disease | Maximum |
|---|---|---|
| | Incidence | Score |
| UNT | 100% (10/10) | 1.9 (+/−0.2) |
| BLK PAR | 100% (11/11) | 1.5 (+/−0.3) |
| PS SOL | 83% (10/12) | 0.8 (+/−0.2) |
| PS PAR | 25% (3/12) | 0.2 (+/−0.1) |

BLK PAR = blank particle;
PS SOL = free PS;
PS PAR = particle containing PS.

While not being bound to any theory, administering certain phospholipids, including phosphatidylserine (PS), can ameliorate symptoms of multiple sclerosis by activation and inducing apoptosis of autoreactive T cells. Particles containing PS can promote the release of a potent immunoregulatory cytokine, TGF-β, from innate immune cells.

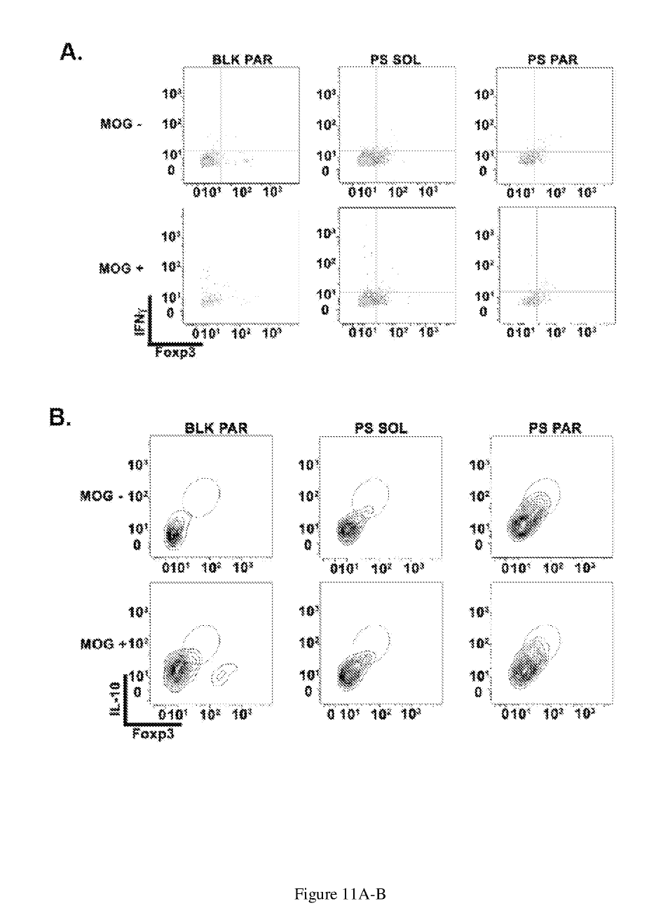

In embodiments, the subject matter disclosed herein is directed to methods of inducing T_{reg} population by administering to a subject an effective amount of particles disclosed herein. As used herein, the term “T_{reg}” refers to regulatory T cells that are a subpopulation of T cells which modulate the immune system. They are also known as suppressor T cells. In particular, these T_{reg} cells maintain tolerance to self antigens and abrogate autoimmune diseases. When self/non-self discrimination fails, the immune system destroys cells and tissues of the body and as a result causes autoimmune diseases. Regulatory T cells actively suppress activation of the immune system and prevent pathological self-reactivity, i.e. autoimmune disease. T_{reg} cells can suppress the immune response of other cells. The particles described herein can induce T_{reg} population by 5% or more. In embodiments, the particles described herein can induce T_{reg} population by 10, 15, 20, 25, 30, 35, 40, 45, 50, 55, 60, 65, 70, 75, 80, 85, 90, 95% or more. The induction can be two-fold, three-fold, four-fold, five-fold and more. Certain effects of the particles on T_{reg} population are shown in FIG. 10A-C and FIG. 11A-B. Biomarkers such as IL-10 and FOXP3 can identify the in vivo effects of the particles.

In embodiments, the subject matter disclosed herein is directed to methods of inducing apoptosis of autoreactive T cells by administering to a subject an effective amount of particles disclosed herein.

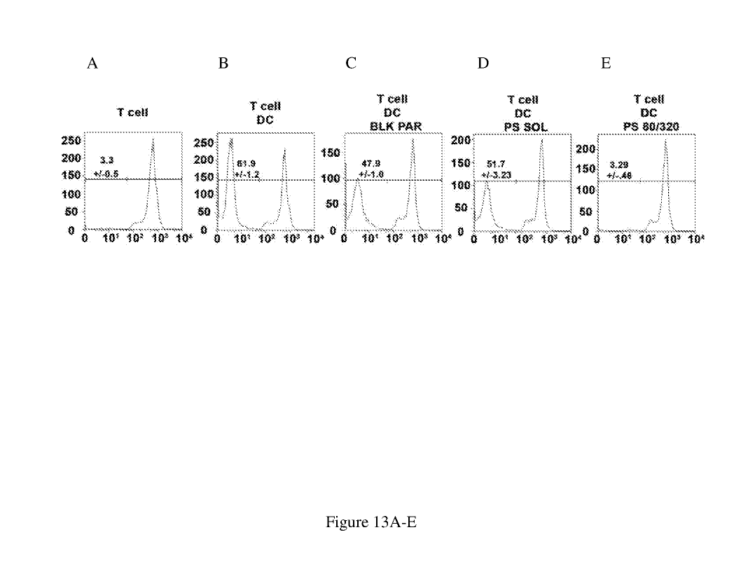

In embodiments, the subject matter disclosed herein is directed to methods of modulating effector T cell proliferation by administering to a subject an effective amount of particles disclosed herein. Memory T cells are a subset of antigen-specific T cells that persist long-term after an infection has resolved. They quickly expand to large numbers of effector T cells upon re-exposure to their cognate antigen, thus providing the immune system with memory against past infections. FIG. 13A-E provide data showing the modulation of effector T cell proliferation.

Specific embodiments include:

1. A micro- or nanoparticle comprising:

a polymer matrix; and

one or more agents selected from the group consisting of phospholipids and fatty acids and combinations thereof.

2. The particle of embodiment 1, wherein the one or more agents are phospholipids.

3. The particle of embodiment claim 2, wherein the phospholipid is a phosphoglyceride.

4. The particle of embodiment 2, wherein the phosphoglyceride is phosphatidylserine.

5. The particle of embodiment 2, wherein the phospholipids are present in an amount of about 1 to about 1,000 μM per particle.

6. The particle of embodiment 2, wherein the phospholipids are present in an amount of about 1 to about 100 μM per particle.

7. The particle of embodiment 2, wherein the phospholipids are present in an amount of about 5 to about 75 μM per particle.

8. The particle of embodiment 2, wherein the phospholipids are present in an amount of about 10 to about 60 μM per particle.

9. The particle of embodiment 2, wherein the phospholipids are present in an amount of about 12.5, 25 or 50 μM per particle.

10. The particle of embodiment 1, wherein the polymer matrix comprises a biodegradable and bioresorbable polymer.

11. The particle of embodiment 10, wherein the polymer matrix comprises PLGA.

12. The particle of embodiment 11, wherein the PLGA is 85:15.

13. The particle of embodiment 11, wherein the PLGA has a molecular weight of about 25,000 to about 100,000.

14. The particle of embodiment 11, wherein the PLGA has a molecular weight of about 75,000.

15. A method of treating an autoimmune disease comprising, administering to a subject an effective amount of the particles of embodiment 1.

16. The method of embodiment 15, wherein the autoimmune disease is an organ specific autoimmune disease.

17. The method of embodiment 15, wherein the autoimmune disease is selected from the group consisting of thyroiditis, insulitis, insulin-dependent diabetes mellitus, multiple sclerosis, iridocyclitis, uveitis, orchitis, hepatitis, Addison's disease, inflammatory bowel diseases, Crohn's disease, ulcerative colitis, myasthenia gravis, rheumatoid arthritis, juvenile arthritis, systemic lupus erythematosus and allergic reactions.

18. The method of embodiment 16, wherein the autoimmune disease is multiple sclerosis.

19. The method of embodiment 18, wherein the multiple sclerosis is primary progressive.

20. The method of embodiment 15, wherein the administration is pulmonary.

21. A method of modulating an immune response by administering an effective amount of the particles of embodiment 1.

22. A method of preparing the particle of embodiment 1 comprising:

i. Contacting a mold with a loading solution, wherein the loading solution comprises about 5% to about 50% (w/w) phospholipid and/or fatty acid and a polymer;

ii. Allowing the loading solution to cure; and

iii. Harvesting the particle.

23. The method of embodiment 22, wherein the loading solution comprises about 5 to about 25% (w/w) phospholipid.

24. The method of embodiment 22, wherein the loading solution comprises about 10% (w/w) phospholipid.

25. The method of embodiment 24, wherein the phospholipid is phosphatidylserine.

26. A particle comprising,

i. a matrix comprising PLGA (85:15), having a molecular weight of about 75,000; and

ii. phosphatidylserine, in an amount of from about 5 to about 55 μM.

The term “treating” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. As used herein the terms “treating” includes “ameliorating,” which refers to all processes wherein there may be a slowing, interrupting, arresting, or stopping of the progression of the condition or symptoms and does not necessarily indicate a total elimination of the underlying condition. In embodiments, the term “ameliorating” and “dampening” refer to a lessening of the severity of a symptom and there are clinical assessments and markers that can be used to identify and quantify the lessening of symptoms. Also included in the amelioration of symptoms is the perception by the subject that the symptoms have lessened.

The term “therapeutically effective amount” as used herein refers to an amount of the plurality of monodisperse particles sufficient to achieve a certain outcome, such as to modulate an immune response in the subject. By “modulating an immune response” is intended an induction of a specific immune response (or immunogenic response) or a regulatory response as opposed to an effector response or dampening an inflammatory response. The effective amount and dosage of such active agents required to be administered for effective treatment are known in the art or can be readily determined by those of skill in this field. Of course, the amount of active agent administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art coupled with the general and specific examples disclosed herein. Where active agents do not have a known dosage for certain diseases, the effective amount of active agent and the amount of a particular dosage form required to be administered for effective treatment can be readily determined by those of skill in this field. Thus, the term “therapeutically effective amount” can mean an amount of a particles or active agent(s) within the particles that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. A “therapeutically effective amount” of a particles or active agent(s) within the particles also means a nontoxic but sufficient amount of the agent to provide the desired effect.

For the prevention or treatment of an automimmune disease, the appropriate dosage will depend on the disease to be treated, as defined above, the severity and course of the disease, whether particles are administered for preventive or therapeutic purposes, previous therapy, the patient's clinical history and response to the protein, and the discretion of the attending physician. The particles are suitably administered to the patient at one time or over a series of treatments. Depending on the type and severity of the disease, about 1 μg/kg to 50 mg/kg (e.g. 0.1-20 mg/kg) of particles is an initial candidate dosage for administration to the subject, whether, for example, by one or more separate administrations, or by continuous infusion. In some embodiments, the dosage of the particles will be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week or every three weeks (e.g. such that the subject receives from about two to about twenty, e.g. about six doses of the protein).

The amount of active agent present in the pharmaceutical composition will depend on the agent. Most useful agents are indicated for certain diseases and conditions and the dose amount of active agent can be readily determined and a pharmaceutical composition comprising the desired amount can be prepared as disclosed herein. Useful values of active agents are from about 1 mg to about 1,500 mg active agent per dosage form of the pharmaceutical composition. Preferred values are from about 100 mg to about 800 mg.

In addition to a phospholipid and/or bioactive fatty acid, the particles described herein can further comprise a biologic or therapeutic agent or drug. The term “therapeutic,” “therapeutic agent,” “active agent,” “active pharmaceutical agent” or “drug” as used herein means any active pharmaceutical ingredient (“API”), including its pharmaceutically acceptable salts (e.g. the hydrochloride salts, the hydrobromide salts, the hydroiodide salts, and the saccharinate salts), as well as in the anhydrous, hydrated, and solvated forms, in the form of prodrugs, and in the individually optically active enantiomers of the API as well as polymorphs of the API. Therapeutic agents include pharmaceutical, chemical or biological agents. Additionally, pharmaceutical, chemical or biological agents can include any agent that has a desired property or affect whether it is a therapeutic agent. For example, agents also include diagnostic agents, biocides and the like. Preferred biological agents include antibodies, proteins and fragments thereof that complement the immunomodulation of the phospholipid and/or bioactive fatty acid. In embodiments, the particles also comprise a known adjuvant that modulates an immune response. Also included are known anti-inflammatory drugs, including natural anti-inflammatory agents, steroids and NSAIDs. These drugs include Alclofenac; Alclometasone Dipropionate; Algestone Acetonide; Alpha Amylase; Amcinafal; Amcinafide; Amfenac Sodium; Amiprilose Hydrochloride; Anakinra; Anitrazafen; Apazone; Balsalazide Disodium; Bendazac; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Difluprednate; Diftalone; Dimethyl Sulfoxide; Drocinonide; Endrysone; Enlimomab; Enolicam Sodium; Etodolac; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Fluocortin Butyl; Fluorometholone Acetate; Fluquazone; Fluretofen; Fluticasone Propionate; Furaprofen; Furobufen; Halcinonide; Halobetasol Propionate; Halopredone Acetate; Ibuprofen; Ibuprofen Aluminum; Ibuprofen Piconol; Ilonidap; Indomethacin Sodium; Indomethacin; Indoprofen Indoxole; Intrazole; Isoflupredone Acetate; Isoxepac; Isoxicam; Ketoprofen; Lomoxicam; Loteprednol Etabonate; Meclofenamate Sodium; Meclofenamic Acid; Meclorisonc Dibutyrate; Mesalamine; Meseclazone; Methylprednisolone Suleptanate; Morniflumate; Nabumetone; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Piroxicam; Piroxicam Cinnamate; Pirprofen; Prednazate; Prednisolone Sodium Phosphate; Prifelone; Prodolic Acid; Proquazone; Rimexolone; Romazarit; Salnacedin; Seclazone; Sermetacin; Sudoxicam; Sulindac; Suprofen; Talniflumate; Tenidap; Tenidap Sodium; Tenoxicam; Tesicam; Tesimide; Tixocortol Pivalate; Tolmetin; Tolmetin Sodium; Triclonide; Triflumidate; and Zidometacin.

Mention is made of the particle's ability to direct Th17 responses. This is particularly effective against fungal infections. Fungal infections include an infection by an organism selected from the species Aspergillus flavus; Aspergillus fumigatus; Aspergillus nidulans; Aspergillus niger; Aspergillus parasiticus; Aspergillus terreus; Blumeria graminis; Candida albicans; Candida cruzei; Candida glabrata; Candida parapsilosis; Candida tropicalis; Colletotrichium trifolii, Cryptococcus neoformans; Encephalitozoon cuniculi; Fusarium graminarium; Fusarium solani; Fusarium sporotrichoides; Histoplasma capsulata; Leptosphaeria nodorum; Mycosphaerella graminicola; Phytophthora capsici; Phytophthora infestans; Plasmopara viticola; Pneumocystis jiroveci; Puccinia coronata; Puccinia graminis; Pyricularia oryzae; Pythium ultimum; Rhizoctonia solani; Trichophyton interdigitale; Trichophyton rubrum; and Ustilago maydis. In this aspect, the phospholipid and/or fatty acid containing particles can further comprise an antifungal agent, e.g., azoles, diazoles, triazoles, miconazole, fluconazole, ketoconazole, clotrimazole, itraconazole griseofulvin, ciclopirox, amorolfine, terbinafine, Amphotericin B and potassium iodide.

The particles can be formulated into pharmaceutical compositions as described herein.

The administration of the particles and compositions comprising the particles can be accomplished through any route known in the art. Routes of administration include intravenous or parenteral administration, oral administration, topical administration, transmucosal administration and transdermal administration. For intravenous or parenteral administration, i.e., injection or infusion, the composition may also contain suitable pharmaceutical diluents and carriers, such as water, saline, dextrose solutions, fructose solutions, ethanol, or oils of animal, vegetative, or synthetic origin. It may also contain preservatives, and buffers as are known in the art. When a therapeutically effective amount is administered by intravenous, cutaneous or subcutaneous injection, the solution can also contain components to adjust pH, isotonicity, stability, and the like, all of which is within the skill in the art. The pharmaceutical composition of the present invention may also contain stabilizers, preservatives, buffers, antioxidants, or other additive known to those of skill in the art.

In particular, aerosolized medicaments are used to deliver particles to the lungs by having the patient inhale the aerosol through a tube and/or mouthpiece coupled to the aerosol generator. By inhaling the aerosolized medicament, the patient can quickly receive a dose of medicament in the lungs. In this way, the particles are delivered in a manner that can be the most efficient for licensing immunity. Aerosols of solid particles comprising the phospholipid and/or bioactive fatty acid may be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the anti-malarial compound, a suitable powder diluent, such as lactose, and an optional surfactant. A second type of illustrative aerosol generator comprises a metered dose inhaler. Metered dose inhalers are pressurized aerosol dispensers, typically containing a suspension or solution formulation of the anti-malarial compound in a liquified propellant. During use these devices discharge the formulation through a valve, adapted to deliver a metered volume, from 10 to 22 microliters to produce a fine particle spray containing the anti-malarial compound.

Suitable propellants include certain chlorofluorocarbon (compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents. Any propellant may be used in carrying out the present invention, including both chlorofluorocarbon-containing propellants and non-chlorofluorocarbon-containing propellants. Fluorocarbon aerosol propellants that may be employed in carrying out the present invention including fluorocarbon propellants in which all hydrogen are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. A stabilizer such as a fluoropolymer may optionally be included in formulations of fluorocarbon propellants, such as described in U.S. Pat. No. 5,376,359 to Johnson.

In pulmonary delivery in particular, therapeutics must circumvent the lung's particle clearance mechanisms such as mucociliary transport, phagocytosis by macrophages and rapid absorption of drug molecules into the systemic circulation. Mucociliary clearance can be reduced by avoiding particle deposition in the tracheobronchial region which contains the cilia and goblets cells that make up the mucociliary escalator. Upon delivery to the pulmonary region, particles can be rapidly cleared by alveolar macrophages.

Typically, compositions for intravenous or parenteral administration comprise a suitable sterile solvent, which may be an isotonic aqueous buffer or pharmaceutically acceptable organic solvent. The compositions can also include a solubilizing agent as is known in the art if necessary. Compositions for intravenous or parenteral administration can optionally include a local anesthetic to lessen pain at the site of the injection. Generally, the ingredients are supplied either separately or mixed together in unit dosage form in a hermetically sealed container such as an ampoule or sachette. The pharmaceutical compositions for administration by injection or infusion can be dispensed, for example, with an infusion bottle containing, for example, sterile pharmaceutical grade water or saline. Where the pharmaceutical compositions are administered by injection, an ampoule of sterile water for injection, saline, or other solvent such as a pharmaceutically acceptable organic solvent can be provided so that the ingredients can be mixed prior to administration.

The duration of intravenous therapy using the pharmaceutical composition of the present invention will vary, depending on the condition being treated or ameliorated and the condition and potential idiosyncratic response of each individual mammal. The duration of each infusion is from about 1 minute to about 1 hour. The infusion can be repeated as necessary.

Systemic formulations include those designed for administration by injection, e.g., subcutaneous, intravenous, intramuscular, intrathecal or intraperitoneal injection. Useful injectable preparations include sterile suspensions, solutions or emulsions of the active compound(s) in aqueous or oily vehicles. The compositions also can contain solubilizing agents, formulating agents, such as suspending, stabilizing and/or dispersing agent. The formulations for injection can be presented in unit dosage form, e.g., in ampules or in multidose containers, and can contain added preservatives. For prophylactic administration, the compound can be administered to a patient at risk of developing one of the previously described conditions or diseases. Alternatively, prophylactic administration can be applied to avoid the onset of symptoms in a patient suffering from or formally diagnosed with the underlying condition.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art coupled with the general and specific examples disclosed herein.

Oral administration of the composition or vehicle can be accomplished using dosage forms including but not limited to capsules, caplets, solutions, suspensions and/or syrups. Such dosage forms are prepared using conventional methods known to those in the field of pharmaceutical formulation and described in the pertinent texts, e.g., in Remington: The Science and Practice of Pharmacy (2000), supra.

The dosage form may be a capsule, in which case the active agent-containing composition may be encapsulated in the form of a liquid. Suitable capsules may be either hard or soft, and are generally made of gelatin, starch, or a cellulosic material, with gelatin capsules preferred. Two-piece hard gelatin capsules are preferably sealed, such as with gelatin bands or the like. See, for e.g., Remington: The Science and Practice of Pharmacy (2000), supra, which describes materials and methods for preparing encapsulated pharmaceuticals.

Capsules may, if desired, be coated so as to provide for delayed release. Dosage forms with delayed release coatings may be manufactured using standard coating procedures and equipment. Such procedures are known to those skilled in the art and described in the pertinent texts (see, for e.g., Remington: The Science and Practice of Pharmacy (2000), supra). Generally, after preparation of the capsule, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained-release dosage forms provide for drug release over an extended time period, and may or may not be delayed release. Generally, as will be appreciated by those of ordinary skill in the art, sustained-release dosage forms are formulated by dispersing a drug within a matrix of a gradually bioerodible (hydrolyzable) material such as an insoluble plastic, a hydrophilic polymer, or a fatty compound. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

In embodiments, the particles release at least about 25% of the phospholipid and/or fatty acid within the matrix of the particle within 48 hours of administration. In embodiments, the particles release at least about 50% of the phospholipid and/or fatty acid within the matrix of the particle within 48 hours of administration. In embodiments, the particles release at least about 75% of the phospholipid and/or fatty acid within the matrix of the particle within 48 hours of administration. In embodiments, the particles release at least about 90% of the phospholipid and/or fatty acid within the matrix of the particle within 48 hours of administration. In embodiments, the particles release at least about 95% of the phospholipid and/or fatty acid within the matrix of the particle within 48 hours of administration. In embodiments, the particles release from about 50% to about 100% of the phospholipid and/or fatty acid within the matrix of the particle within 48 hours of administration. In embodiments, the particles release from about 75% to about 95% of the phospholipid and/or fatty acid within the matrix of the particle within 48 hours of administration. In embodiments, the particles release from about 85% to about 90% of the phospholipid and/or fatty acid within the matrix of the particle within 48 hours of administration. In embodiments, the above % releases are within 36 hours after administration. In embodiments, the above % releases are within 24 hours after administration. In embodiments, the above % releases are within 12 hours after administration. In embodiments, the above % releases are within 6 hours after administration. In embodiments, the above % releases are within 4 hours after administration. In embodiments, the above % releases are within 2 hours after administration. In embodiments, the above % releases are within 1 hour after administration.

Topical administration of an agent containing a phosphpolipid and/or fatty acid can be accomplished using any formulation suitable for application to the body surface, and may comprise, for example, an ointment, cream, gel, lotion, solution, paste or the like, and/or may be prepared so as to contain liposomes, micelles, and/or microspheres and/or microneedles. Preferred topical formulations herein are ointments, creams, and gels.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy (2000), supra, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy (2002), supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels-are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Transmucosal administration of an agent containing a phosphpolipid and/or fatty acid can be accomplished using any type of formulation or dosage unit suitable for application to mucosal tissue. For example, the particles containing a phosphpolipid and/or fatty acid can be administered to the buccal mucosa in an adhesive patch, sublingually or lingually as a cream, ointment, or paste, nasally as droplets or a nasal spray, or by inhalation of an aerosol formulation or a non-aerosol liquid formulation.

Preferred buccal dosage forms will typically comprise a therapeutically effective amount of a phosphpolipid and/or fatty acid and a bioerodible (hydrolyzable) polymeric carrier that may also serve to adhere the dosage form to the buccal mucosa. The buccal dosage unit is fabricated so as to erode over a predetermined time period, wherein drug delivery is provided essentially throughout. The time period is typically in the range of from about 1 hour to about 72 hours. Preferred buccal delivery preferably occurs over a time period of from about 2 hours to about 24 hours. Buccal drug delivery for short-term use should preferably occur over a time period of from about 2 hours to about 8 hours, more preferably over a time period of from about 3 hours to about 4 hours. As needed buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The “therapeutically effective amount” of an agent in the buccal dosage unit will of course depend on the potency and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol®, which may be obtained from B. F. Goodrich, is one such polymer). Other suitable polymers include, but are not limited to: hydrolyzed polyvinylalcohol; polyethylene oxides (e.g., Sentry Polyox® water soluble resins, available from Union Carbide); polyacrylates (e.g., Gantrez®, which may be obtained from GAF); vinyl polymers and copolymers; polyvinylpyrrolidone; dextran; guar gum; pectins; starches; and cellulosic polymers such as hydroxypropyl methylcellulose, (e.g., Methocel®, which may be obtained from the Dow Chemical Company), hydroxypropyl cellulose (e.g., Klucel®, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components may also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone® XL, which may be obtained from GAF), cross-linked carboxylic methylcelluloses, such as croscarmelose (e.g., Ac-di-sol®, which may be obtained from FMC), alginic acid, and sodium carboxymethyl starches (e.g., Explotab®, which may be obtained from Edward Medell Co., Inc.), methylcellulose, agar bentonite and alginic acid. Suitable diluents are those which are generally useful in pharmaceutical formulations prepared using compression techniques, e.g., dicalcium phosphate dihydrate (e.g., Di-Tab®, which may be obtained from Stauffer), sugars that have been processed by cocrystallization with dextrin (e.g., co-crystallized sucrose and dextrin such as Di-Pak®, which may be obtained from Amstar), calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include creams, ointments and pastes. The cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units are fabricated to disintegrate rapidly. The time period for complete disintegration of the dosage unit is typically in the range of from about 10 seconds to about 30 minutes, and optimally is less than 5 minutes.

Other components may also be incorporated into the sublingual and lingual dosage forms described herein. The additional components include, but are not limited to binders, disintegrants, wetting agents, lubricants, and the like. Examples of binders that may be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy (2000), supra).

Other preferred compositions for sublingual administration include, for example, a bioadhesive; a spray, paint, or swab applied to the tongue; or the like. Increased residence time increases the likelihood that the administered invention can be absorbed by the mucosal tissue.

Transdermal administration of a particle containing a phosphpolipid and/or fatty acid through the skin or mucosal tissue can be accomplished using conventional transdermal drug delivery systems, wherein the agent is contained within a laminated structure (typically referred to as a transdermal “patch”) that serves as a drug delivery device to be affixed to the skin.

Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed below in transmucosal compositions.

Formulations can comprise one or more anesthetics. Patient discomfort or phlebitis and the like can be managed using anesthetic at the site of injection. If used, the anesthetic can be administered separately or as a component of the composition. One or more anesthetics, if present in the composition, is selected from the group consisting of lignocaine, bupivacaine, dibucaine, procaine, chloroprocaine, prilocaine, mepivacaine, etidocaine, tetracaine, lidocaine and xylocaine, and salts, derivatives or mixtures thereof.

The present subject matter is further described herein by the following non-limiting examples which further illustrate the invention, and are not intended, nor should they be interpreted to, limit the scope of the invention.

### EXAMPLES

### 1. Phospholipid Containing Particles

a. Materials

Poly(D,L-lactide-co-glycolide) (lactide:glycolide 85:15, 0.65 dL/g Inherent Viscosity at 30° C.) was purchased from Sigma-Aldrich. Chloroform and solvents (acetonitrile and water) for high performance liquid chromatography (HPLC) were purchased from Fisher Scientific. Docosahexaenoic acid (DHA) was purchased from Cayman Chemicals. Phosphatidylserine (PS) (brain, porcine) was purchased from Avanti Polar Lipids. Poly(ethylene terephthalate) (PET) sheets (6″ width) were purchased from KRS plastics. Fluorocur®, d=80 nm; h=320 nm; prefabricated molds and 2000 g/mol polyvinyl alcohol (PVOH) coated PET sheets were provided by Liquidia Technologies.

b. Particle Fabrication

PS and PLGA were dissolved separately in chloroform. The solutions of PS and

PLGA were mixed at ratios of 10:90 (PS:PLGA), and the sample was diluted to 2 wt % (mass/mass) solution with chloroform. A thin film of DHA or PS and PLGA was deposited on a 6″×12″ sheet of PET by spreading 200 μL of solution using a #5 Mayer Rod (R.D. Specialties). The solvent was evaporated with heat. The PET sheet with the film was then placed in contact with the patterned side of a mold and passed through heated nips (ChemInstruments Hot Roll Laminator) at 130° C. and 80 psi. The mold was split from the PET sheet as they both passed through the hot laminator. The patterned side of the mold was then placed in contact with a sheet of PET sheet coated with 2000 g/mol PVOH. This was then passed through the hot laminator to transfer the particles from the mold to the PET sheet. The mold was then peeled from the PET sheet. The particles were removed by passing the PVOH coated PET sheet through motorized rollers and applying water to dissolve the PVOH to release the particles. To remove excess PVOH, the particles were purified and then concentrated by tangential flow filtration (Spectrum Labs).

c. Particle Characterization

Particles were imaged by scanning electron microscopy (SEM) by pipetting a 3 μL sample of particle on a glass slide. The sample was then dried and coated with 3 nm gold palladium alloy using a Cressington 108 auto sputter coater. Images were taken at an accelerating voltage of 2 kV using a Hitachi model S-4700 SEM. For size and zeta potential measurement, dynamic light scattering (DLS) (Malvern Instruments Nano-ZS) was used.

d. Drug Loading

DHA or PS was measured using an Agilent Technologies Series 1200 HPLC with a C18 reverse phase column (Zorbax Eclipse XDB-C18, 4.6×100 mm, 3.5 micron). A linear gradient from 85:15 of methanol with 0.1% trifluoroacetic acid (TFA): water with 0.1% TFA to 100% methanol with 0.1% TFA was run over 25 minutes. The flow rate was 1 mL/min and an ELSD detector was used for quantification. Particle samples were prepared by diluting the sample with a 50:50 acetonitrile:water solution and mixing the sample to break down the particle and dissolve the PVOH. Standards of PS were prepared in 50:50 acetonitrile:water.

### 2. Induction of Regulatory T Cells

Using a co-culture model with mouse dendritic cells and 2D2 T cells specific for the MOG-peptide, Th1-skewed IFN-γ producing T cells were turned into FOXP3+, IL-10 producing T regulatory cells (FIGS. 10 and 11). In the absence of antigen, roughly 20% of all T cells were programmed into anti-inflammatory IL-10 producing FOXP3+ regulatory T cells. This was about a 10-15 fold increase as compared to blank PLGA particles controls. This was about 4-5 fold increase over the soluble PS control at the same dose. This is an exceptionally high % of T regulatory cells in any in vitro or in vivo system. As this was in the absence of antigen, this suggests PS-PLGA particles can be used to program tolerant immune environments throughout the body; a potentially transformative clinical treatment option

In the presence of soluble MOG antigen, about 10% of T cells produced IFN-γ in the blank 80×320 nm PLGA control group. IFN-γ was inhibited to baseline levels when PS was delivered in either soluble or particulate form. The presence of antigen led to a 10% IL-10+, FOXP3+ T regulatory population in the presence of blank PLGA particles or soluble PS. PS-PLGA particles induced 2 fold higher levels of this regulatory T cell population, about 20% of all T cells. These results show that PS-PLGA particles can induce immune tolerance in the presence or absence of Th1 skewed antigens.

These data reveal emergent properties of PS particles. At the same dose, soluble PS can inhibit IFN-γ as well as particulate PS, but only particulate PS can robustly induce IL-10+ and FOXP3+ T cells.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1-15. (canceled)

16. A method for inducing T_{reg} population comprising:
administering a plurality of particles to a patient in need thereof, wherein each particle of the plurality comprises:
a matrix comprising poly(D,L-lactide-co-glycolide) (PLGA) comprising a molar ratio of lactide:glycolide of about 85:15 and an inherent viscosity of about 0.65 dL/g at 30° C., and phosphatidylserine (PS); wherein the weight percent of PS is about 2 weight percent to about 25 weight percent;
a shape comprising an aspect ratio greater than 2:1; and
wherein said inducing a T_{reg} population comprises at least a two-fold increase in T_{reg} population compared to T_{reg} population induction from an equivalent concentration of soluble PS.

17. The method of claim 16, wherein the weight percent of PS is about 5 weight percent to about 15 weight percent.

18. The method of claim 16, wherein the weight percent of PS is about 10 weight percent.

19. The method of claim 16, wherein administration is to treat an autoimmune disease.

20. The method of claim 19, wherein the autoimmune disease is selected from the group consisting of thyroiditis, insulitis, insulin-dependent diabetes mellitus, multiple sclerosis, iridocyclitis, uveitis, orchitis, hepatitis, Addison's disease, inflammatory bowel diseases, Crohn's disease, ulcerative colitis, myasthenia gravis, rheumatoid arthritis, juvenile arthritis, systemic lupus erythematosus, and allergic reactions.

21. The method of claim 16, wherein the shape comprises a first dimension of less than about 200 nm and a second dimension greater than about 200 nm.

22. The method of claim 16, wherein the shape comprises a dimension of less than about 100 nm.

23. The method of claim 16, wherein the shape comprises a diameter of about 80 nm and a length of about 320 nm.

24. The method of claim 16, wherein administration is via a route selected from the group consisting of intravenous, parenteral, oral, topical, transmucosal, transdermal, inhalation, and injection.

25. The method of claim 24, wherein the injection route is selected from the group consisting of subcutaneous, intramuscular, intrathecal, and intraperitoneal.

26. A method for reducing inflammation cytokines, comprising:
administering a plurality of particles to a patient in need thereof, wherein each particle of the plurality comprises:
a matrix comprising poly(D,L-lactide-co-glycolide) (PLGA) comprising a molar ratio of lactide:glycolide of about 85:15 and an inherent viscosity of about 0.65 dL/g at 30° C., and phosphatidylserine (PS); wherein the weight percent of PS is about 2 weight percent to about 25 weight percent; and
a shape comprising an aspect ratio greater than 2:1;
wherein said reducing inflammation cytokines comprises reducing IFN-γ, IL-2, IL-6, and TNF-α inflammation cytokines produced compared to IFN-γ, IL-2, IL-6, and TNF-α cytokines produced in response to an equivalent concentration of soluble PS.

27. The method of claim 26, wherein the weight percent of PS is about 5 weight percent to about 15 weight percent.

28. The method of claim 26, wherein the weight percent of PS is about 10 weight percent.

29. The method of claim 26, wherein the shape comprises a first dimension of less than about 200 nm and a second dimension greater than about 200 nm.

30. The method of claim 26, wherein the shape comprises a dimension of less than about 100 nm.

31. The method of claim 26, wherein the shape comprises a diameter of about 80 nm and a length of about 320 nm.

32. The method of claim 26, wherein administration is to treat an autoimmune disease.

33. The method of claim 32, wherein the autoimmune disease is selected from the group consisting of thyroiditis, insulitis, insulin-dependent diabetes mellitus, multiple sclerosis, iridocyclitis, uveitis, orchitis, hepatitis, Addison's disease, inflammatory bowel diseases, Crohn's disease, ulcerative colitis, myasthenia gravis, rheumatoid arthritis, juvenile arthritis, systemic lupus erythematosus, and allergic reactions.

34. The method of claim 26, wherein administration is via a route selected from the group consisting of intravenous, parenteral, oral, topical, transmucosal, transdermal, inhalation, and injection.

35. The method of claim 34, wherein the injection route is selected from the group consisting of subcutaneous, intramuscular, intrathecal, and intraperitoneal.
